(19) United States
(12) Patent Application Publication
(10) Pub. No.: US 2020/0380404 A1
(43) Pub. Date: Dec. 3, 2020

(54) MEDICAL SUPPORT PREDICTION FOR EMERGENCY SITUATIONS

(71) Applicant: INTERNATIONAL BUSINESS MACHINES CORPORATION, Armonk, NY (US)

(72) Inventors: Sarbajit K. Rakshit, Kolkata (IN); James E. Bostick, Cedar Park, TX (US); Martin G. Keen, Cary, NC (US); John M. Ganci, JR., Raleigh, NC (US)

(21) Appl. No.: 16/427,531

(22) Filed: May 31, 2019

(51) Int. Cl. G06N 20/00; G06F 16/23; G06N 5/04; G16H 40/20; A62B 99/00

(52) U.S. Cl. CPC G06N 20/00; G06F 16/23; A62B 99/00; G16H 40/20; G06N 5/04

(57) ABSTRACT

A method, computer system, and a computer program product for predictive support is provided. Embodiments of the present invention may include creating a knowledge corpus based on historical data. Embodiments of the present invention may include building a machine learning model based on historical data. Embodiments of the present invention may include gathering real-time data from an event site. Embodiments of the present invention may include analyzing the gathered real-time data using the built machine learning model. Embodiments of the present invention may include providing a response to a plurality of users. Embodiments of the present invention may include training the machine learning model based on the analyzed real-time data.

### BACKGROUND

The present invention relates generally to the field of computing, and more particularly to predictive analytics. The ability to access and analyze information from a location or an area in response to an event may be limited by the location. The location of an event may not be a highly populated area, thus, providing relief efforts for emergency related events in locations that may be difficult to access could cause injuries to become more severe and possibly fatal as a response time lengthens.

### SUMMARY

Embodiments of the present invention disclose a method, computer system, and a computer program product for predictive support. Embodiments of the present invention may include creating a knowledge corpus based on historical data. Embodiments of the present invention may include building a machine learning model based on historical data. Embodiments of the present invention may include gathering real-time data from an event site. Embodiments of the present invention may include analyzing the gathered real-time data using the built machine learning model. Embodiments of the present invention may include providing a response to a plurality of users. Embodiments of the present invention may include training the machine learning model based on the analyzed real-time data.

### BRIEF DESCRIPTION OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for predicting support based on a built knowledge corpus and machine learning according to at least one embodiment;

FIG. 3 is a block diagram of internal and external components of computers and servers depicted in FIG. 1 according to at least one embodiment;

FIG. 4 is a block diagram of an illustrative cloud computing environment including the computer system depicted in FIG. 1, in accordance with an embodiment of the present disclosure; and

FIG. 5 is a block diagram of functional layers of the illustrative cloud computing environment of FIG. 4, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

As previously described, the ability to access and analyze information from a location or an area in response to an event may be limited by the location. The location of an event may not be a highly populated area, thus, providing relief efforts for emergency related events in locations that may be difficult to access could cause injuries to become more severe and possibly fatal as a response time lengthens. Natural disasters, accidents, emergencies, catastrophes or other events may cause people to become injured and possibly stranded after an injury has occurred. For example, an earthquake, a tornado, a hurricane, a tropical storm, a collapsing bridge, a capsized boat or a hiking accident may become an injury related event that leaves some people stranded, some injured and some lost.

When an injury related event occurs in a location that is difficult to access, time and access to information at the location or site of the event becomes a critical element of helping the injured or stranded individuals. At disaster sites, medical facilities may be setup near the site to provide support. The number of facilities needed for support and facility types may not be readily known. Therefore, it may be advantageous to, among other things, quickly evaluate and analyze an event location to assist injured or stranded individuals by quickly assessing the scene, identifying injuries, leveraging the proper emergency personnel, providing proper first responders and organizing medical support that aligns with the needs of the injured individuals.

The following described exemplary embodiments provide a system, method and program product for predicting optimal medical support related to an event. As such, embodiments of the present invention have the capacity to improve the technical field of predictive analytics by improving the response time to analyze and provide support at an injury related event. Quick access to a location that has incurred a crisis situation can help to provide proper search, rescue and medical support to injured individuals. More specifically, an improved response time and corresponding predictive analysis of retrieved information is created using augmented reality to predict the prioritization of medical needs based on an injury analysis. A prediction is made by creating a knowledgebase, incorporating historical data, gathering real-time data using various devices and analyzing the combined data. The various devices may reach secluded, difficult or dangerous locations and transmit information for analysis before emergency personnel can reach the location to assist.

According an embodiment, augmented intelligence (AI) and may be used to visualize an event and prioritize a process for individuals at the event to keep safe, to reach safety or for emergency medical personnel to assist with proper support. The present embodiment may be used for purposes other than crisis situations, such as monitoring a concert, a sporting event, searching for a missing person in a secluded area, monitoring an offshore oil rig or monitoring any event that a large amount of people attend. The use case example in the present embodiment may include a disaster site that is difficult for emergency medical personnel to reach and has caused multiple injuries. The disaster site may be created by, for example, a collapsed bridge, an earthquake, a wildfire or a flood.

According to an embodiment, multiple devices are configured to communicate with the predictive support program via a central server, a central processing system, a central repository or an artificial intelligence (AI) system. The central repository may include a database from where an AI system pulls information from for analysis. The AI system may include, for example, IBM Watson® (IBM Watson and all IBM Watson-based trademarks and logos are trademarks or registered trademarks of International Business Machines Corporation and/or its affiliates). The multiple devices may be configured to transmit data to the central repository and to receive data for transmission or processing. A knowledge corpus may be created using both historical data and real-time data. The historical data may be used to train a machine learning model, or a deep learning model and the real-time data may be used to further train the models. Real time processing and analysis of the received data using augmented reality or augmented intelligence may provide an output of data to help provide support to the injured individuals.

The gathered data may allow the proper authorities to quickly learn the number of injuries, the severity of injuries, the types of injuries and the correct emergency service units that may be needed to assist the injured individuals. The gathered data is analyzed using an initially created knowledgebase, historical data relating to the event, augmented intelligence, natural language processing, semantic analysis, sentiment analysis, machine learning and predictive analytics.

Devices may include computing devices capable of gathering images, audio content, video content, biometric content, storing data or transferring data over a communication network. Devices may include, for example, cameras, microphones, internet of things (IoT) devices, sensors, smart phones, smart watches, smart tablets, personal computers, automotive devices, augmented reality devices, smart glasses, virtual reality headsets, medical devices or unmanned devices that may be used on land, by sea or by air.

Devices with cameras may capture images or a video feed to visualize the disaster site and devices with microphones may capture sounds or an audio feed of the site. Devices with cameras, microphones and the ability to capture biometric data may include, for example, a smart phone or a smart watch. Devices with sensors and the capability to transmit real-time data may include, for example, IoT devices, smart phones and smart watches. Computerized or robotic devices that are not attached to an individual may capture data at the site using capabilities such as cameras, microphones, sensors and the transmission of data. The computerized or robotic devices may assist in reaching areas of the disaster site before the first responders get to the location or reaching areas of the disaster site that first responders may not be able to access. If a first responder reaches the site before a robotic device, then the first responders may, for example, be wearing smart glasses or an augmented reality device to transmit real-time data of the disaster site to the central server and database for processing.

For example, a natural disaster site may be broadly defined as any emergency situation that may require the assistance of emergency medical personnel, such as a collapsed bridge. The first responders may arrive, assess the situation and begin to organize how to assist the injured individuals, starting with the most severely injured. Responders may make immediate decisions to help one person versus another person based on the injuries and the number of available responders. The predictive support program may aid in the assessing, analyzing and organizing process while the responders are at the scene or before the responders get to the scene.

An additional method to obtain data simultaneously, in real-time during or quickly after an event such as a natural disaster may include the use of IoT devices and other devices near the site during the event to transmit data to the central repository. For example, sensors or cameras on a bridge, a personal wearable device worn by an individual during the event or a personal device near an individual during the event. The ability to assess the site as quickly as possible may assist the authorities to dispatch the proper responders for the injured individuals. For example, in the event of a bridge collapsing, IoT devices and sensors attached to the bridge, the automobile devices, biometric devices and smart devices with the injured or stuck individuals may all transmit data to the central repository for a police official to evaluate. The proper authority may immediately learn, via the incoming data, that various different medical attention is needed that corresponds to a bridge collapse.

Evaluations may be made regarding the prioritization of injuries based on how life threatening the injuries are. Additionally, information may be transmitted in real time that may relate to alternative individual conditions that would not be predicted by medical personnel during a bridge collapse, such as a stranded individual suffering from anxiety, someone on the way to a hospital to give birth, someone in diabetic shock, someone suffering from claustrophobia and someone having an allergic reaction. These additional real-time evaluations may not have been predicted by a natural disaster situation, but the additional real-time evaluations may be life threatening and avoided. An unmanned robotic device that can drive via remote on uneven terrain or an unmanned aerial vehicle may have the ability to deliver medicine quickly to a person having an allergic reaction, thus, performing a lifesaving task that would otherwise may not have been identified.

Devices are configured to communicate with the central repository, applications and services associated with the central repository. Devices may be configured via an internet protocol (IP) address, an application username and password, an application available for free public use or a software as a service (SaaS) application. Emergency services personnel, government services personnel or first responders to an event may configure a wearable device, such as augmented reality glasses with a camera or a microphone or a pin with a camera and a microphone attached to a uniform to transmit data the central server and repository.

Individuals or users may also register devices to the central repository via an application and a service for future potential events. The pre-registration to the central repository application may provide data from a user device to be transmitted automatically to the central server for processing during an event. The pre-registration process may gain user permissions for automatic transmission of data when the user is in a disaster event, when the user is in a mapped crisis zone, or when a user is near a disaster site.

According to an embodiment, consent may be obtained from an individual or a user via an opt-in feature or an opt-out feature prior to commencing the collection of data. For example, in some embodiments, the user may be notified when the collection of data begins via a type written message provided on a graphical user interface (GUI) or a screen of a user computing device. According to other embodiments, the user may be notified when the collection of data begins via an audio message and the user may use the audio feature to opt-in or opt-out. In each case, the user operator is provided with a prompt or a notification to acknowledge an opt-in feature or an opt-out feature unless prior consent was given at the pre-registration phase.

An example of prior consent may include a situation or an event that renders the user unconscious and the user pre-consented to have biometric content, audio content and video content that occurred a specified amount of time prior to the event and during the real-time event to be transmitted to the central repository for processing and evaluation. The pre-consent feature may also prompt the user for a current acknowledgement notification, however, if a time period passes with no response is provided or the device is able to immediately determine that the user is unconscious, then the data within a pre-agreed upon time period may be transmitted. The automatic transmission of data or the data transmitted after a secondary acknowledgement permission may include audio feeds, video feeds, images, sounds or biometric data. Users may register one or more user devices to the central repository application.

The knowledge corpus may be created to receive and store domain specific data relating to a particular event, issue, topic or industry. The knowledge may increase over time and thus become more robust, accurate and effective. The knowledge corpus may be known as a corpus, a database, a knowledgebase, a repository or a central storage database.

In an embodiment, the knowledge corpus is created to treat individuals that are in a disaster site. The corpus may gather data, such as historical medical data and previous disaster site data, to begin compiling training data or a training dataset for machine training, data classification and machine learning. For example, previous medical reports from an injured individual from a similar disaster site may be stored in the knowledge corpus and parsed for information. Parsed information may include the type of injury, the type of accident, the severity of the injury, a visual pattern of the injury, the treatment that was provided, the response and movement pattern of the injured individual, when the individual was released, the procedures applied and the voice texture of the injured individual.

The knowledge corpus, as it gathers either more historical data or real-time data, may continue to learn over time based on, for example, images and associated statistics from the location of the site. Additional learning may include specific images with statistics on the type of injury and the type of medical support that may be needed such that a future disaster site is able to more quickly assess the injuries and treatments with a high accuracy.

Data may be gathered prior to, simultaneously, in real-time and after the event. The gathered data may be in the form of, for example, an AI visualization of the disaster site and the visualization may provide pertinent information for authorities, such as the number of injuries, the severity of injuries, the types of injuries and the correct emergency service units that may be dispatched to assist the injured individuals. The received and gathered data may be structured data or unstructured data and may be processed using natural language processing (NLP), semantic analysis and sentiment analysis. Machine learning, deep learning and predictive analytics may also be utilized for data evaluation and continual machine learning.

Structured data may include data that is highly organized, such as a spreadsheet, relational database or data that is stored in a fixed field. Unstructured data may include data that is not organized and has an unconventional internal structure, such as a portable document format (PDF), an image, a presentation, a webpage, video content, audio content, an email, a word processing document or multimedia content. NLP may process the data to extract information that is meaningful to a particular industry or to a particular event, such as extracting information by subject matter or topic. An NLP system may be created and trained by rules or machine learning and word embeddings.

Semantic analysis may be used to infer the meaning and intent of the words and phrases in the data, both verbal and non-verbal. For example, verbal meaning may be inferred using the spoken word and phrases captured by a microphone during communication at the event site or communication between the event site and emergency personnel. Nonverbal meaning may be inferred using words, sentences or phrases identified in text messages, social media postings or emails. Semantic analysis may consider current and historical data associated with a corpus. Current data may be data that is added to a corpus in real-time, for example, via an IoT device, a sensor, a user device or an automobile device. Current data may generally refer to, for example, a video stream or an audio stream of information coming from a disaster site. Historical data may include, for example, electronic book data stored in a library database relating to natural disasters, emergency procedures and medical diagnoses. Semantic analysis may also consider syntactic structures at various levels to infer meaning to words, phrases, sentences and paragraphs scanned from a corpus. Static data may also be considered through semantic analysis, for example, when an augmented reality device receives raw data from software applications and filters the data into meaningful data.

Sentiment analysis may be used to understand how communication may be received by a user or interpreted by an individual the user is communicating with. Sentiment analysis may be processed through, for example, voice identifier software received by a microphone on the augmented reality device, facial expression identifier software received by a camera on smart glasses or by biometric identifier software received by a wearable device such as a smart phone that captures and measures a heartrate or a camera attached to the augmented reality device that measures pupil dilation. Sentiment may also be measured by the tone of voice of the individuals communicating and the syntactic tone in written messages, such as text messages, emails and social media posts.

NLP algorithms may use data in a corpus as a source to scan the data for pre-defined keywords that may be used for each event or subject matter. Machine learning may be incorporated by, for example, analyzing medical journals, medical injuries, natural disasters, natural disaster protocols, legislative policy data, hospital guidelines, government guidelines or emergency protocols. Data may be mined from various copra for machine learning. Data mining may include a process of extracting structured and unstructured data from larger datasets. Datasets may be stored on a database or a corpus and data may be mined for specific events, domains or industries. Industry specific corpora datasets and data may include, for example, telecommunication data, medical data, financial data, legal data, legislative data, business data, transportation data, agriculture data or industrial data.

According to an embodiment, the predictive support program may use image analysis to gather event site data and to train the machine learning model to search for clues of individuals, for example, that may have been subdued under a fallen infrastructure. The model may be trained to seek potential hidden features that may assist, for example, in search and rescue missions. The machine learning model may be trained to search for and identify various types of injuries and diagnoses. The predictions created from the machine learning model, for example, related to injuries at an event site, may also prioritize a process for treating the injuries and create a priority-based list for the first responders or emergency personnel to treat the injured people in the order of severity. Real-time audio streaming or audio recordings may be transmitted from the event site to the central server for speech to text recognition or NLP processing to find clues that may be missed by first responders, such as an individual unseen and unheard by a first responder under rubble. For example, a microphone and a global positioning system (GPS) may pick up human chatter or a location under the rubble.

The predictive support program may count the injuries at a site and map the total site area to verify an accurate count of the injured individuals using, for example, mapping software and image analysis based on the input data. Various cameras may obtain images and videos from more than one site location, therefore, creating the ability to dynamically identify the totality of the site and identify the medical services that may be needed. The medical services may include one or more on-site medical booths, the number of ambulances, the number of fire trucks, the number of first responders, the number of medical professionals, and the number of administrative personnel professionals for site organization.

The boundaries of the site may be identified and, for example, drawn on a map. If a user has configured or registered the device, then the registered device may provide a GPS signal to indicate that the user is within a drawn boundary. Images, audio, video, social media data or text messages may be acknowledged and approved by the user to be transmitted to the central repository.

Incoming data during an event may be captured by the central system for the disaster site area and a feature of the predictive support program may also block data outside the disaster site from entering the central system for processing. For example, one camera may view and record data 5 feet to the West and then the camera may be blocked by a structure. At the same time, the camera may view and record data 7 feet to the North, 4 feet to the South and 16 feet to the East. Image analysis may be processed based on the available camera views and the processed image analysis for this particular camera may be identified on a map as content that has been seen, captured, accounted for or analyzed. The number of injured individuals within this camera view may be assessed. For example, image analysis may discover that images indicate that within this zone, 14 people are on the ground and 6 of the 14 people are being attended to by first responders. Image analysis may also consider the severity of each injury by rating the severity. Each injury may be processed by the central server, the knowledge corpus and the predictive support program to identify each injury and to provide treatments. For example, an image of an individual on the ground holding a left ankle may be rated as less severe than a person who is trapped underneath rubble. The initial assessment of the number of injuries and the types of injuries within a seen or captured range on the map may be counted.

Other cameras may obtain and transmit data from other areas within the designated boundary of an event site. If an overlap camera angle captures data within an area that has already been accounted for, then the predictive support program may recount or recheck the area. A recount or recheck of the area may also be processed if a certain time period has passed. For example, a threshold amount of time may be set for a recount based on historical data of similar disaster sites and based on the various injuries and treatments occurring. If a count is made in a duplicated area, then the count may be considered a replacement count.

For sections that may not overlap, the section will be counted and blocked or identified as seen or captured. For example, if a disaster site has 3 mapped subsections of various sizes and the first subsection contains 7 total injuries and 2 of the 7 injuries are rated as severe, the second subsection contains 3 injures and the third subsection contains 1 severe injury. Based on the size of the disaster site and the size of the seen subsections, there is a total of 3 severe injuries, 11 total injuries and only 27% of the disaster site has been mapped. The injury numbers may change as the predictive support program tracks more people as data is gathered.

Microphone data may be used to assess the event site and to alter the severity level, for example, as the data is gathered from first responders and from registered users. The data obtained from the microphones may be processed and the total injured and severity ratings may be altered based on the communication between the injured, the injured and the first responders, the injured and an emergency administrator personnel or the first responders and the emergency administrator personnel. Speech to text translation and NLP may be used to identify key phrases and reactions used by individuals. For example, classifiers may be trained to alter the severity of the injuries when communication at an event GPS location includes a phrase stating that someone is losing consciousness. The decibel level of the voice may also alter the severity rating of the injuries by a high and distressed voice being associated with more severe injuries and a less distressed tone and a calm demeanor being associated with less severe injuries.

Biometric data may also be used to assess the event site and to alter the severity level. If allowed by the user and the biometric capabilities are turned on, the registered users may have a smart device that is capable of transmitting heartbeat data, medical data, pulse rate data, blood pressure data or pupil dilation data to the central repository for processing. Severity levels may be altered depending on the flux of biometric data, for example, a lowering pulse rate may increase the severity level. The severity of the injuries may be predicted based on the obtained data and solutions may be provided using a trained model.

The multiple configured devices at and near the scene may begin transmitting data to the central repository and central server for processing and may be analyzed by the trained machine learning models. Individuals at the scene may also, if they have not yet previously registered the devices to the central server, may register at the time of the disaster and begin transmitting data to assess the severity of the scene and injuries.

Initial training of the machine learning model may use, for example, historical data relating to the event. The training model will evolve over time and become more robust with improved accuracy of predictive capabilities. Supervised, semi-supervised and unsupervised machine learning may be used for training purposes. Supervised learning may use a labeled dataset to train a ML model. Unsupervised learning may use all unlabeled data to train a ML model. Semi-supervised learning may use both labeled datasets and unlabeled datasets to train a ML model. Ground truth may be added to the predictive support program, for example, subject matter expert (SME) input to improve the accuracy of the model over time. Subject matter expert input may include, for example, manual input based on medical notes, first examiner reports and doctor treatments, followed by the results of the treatments.

As the number of injuries become identified, the injuries are compared against, for example, available emergency services personnel, the number of on-site medical booths, the number of ambulances, the number of helicopters, the number of fire trucks and the number of nearby emergency hospital facilities. If the number of injuries is greater than the capabilities available, then the priority injured individuals based on severity may be responded to first and the ambulances may be used by the higher priority individuals based on rank or rating. As more responders, emergency medical personnel and ambulances become available, the next level of priority may be responded to. As the number of injured individuals grow, the predictive support program may immediately respond by alerting additional emergency personnel, such as other hospitals, other EMS or other emergency transportation units. The additional numbers may be communicated or transmitted to the proper authorities for distribution of the additional information.

As the machine learning model of the predictive support program is further trained over time, the additional data and images provided may be statistically tracked. Statistical tracking may occur as correlations between the severity of the injuries being weighed against the initially predicted severity of the injuries, thus, levels of severity may be learned and improved over time. The additional or updated data may assist in growing the knowledge corpus, for example, based on other disasters. The injury tracking and treatment suggestions made using the predictive analytics may improve over time and become more developed with treatments that provided optimal recovery time or with certain types of first responders that were best equipped to handle the situation. Additional or follow-up data may be transmitted into the knowledge corpus, for example, by an administrator compiling a report after the incident, by the hospital that treated the specific injuries or by a SME based on disaster recovery expertise. The additional data may be considered ground truth for the machine learning capabilities, improvement or accuracy. The knowledge corpus may assist in combining data for predicting the severity of injuries and the types of injuries that have been captured and identified by the cameras and microphones. The captured images are further analyzed so that the knowledge corpus may correlate visual attributes of different types of injuries, voice textures, movement and response patterns of injured individuals with the severity and the type of injury.

In an embodiment, image and voice analysis may identify an injured person. Once identified and with the proper approvals and acknowledgements, previous statistics and medical data relating to the injured person may be retrieved from a different database. For example, the first responders arrive and are going to assist an injured person that has been identified via image analysis. The medical history of the individual may be quickly obtained and processed. The medical history shows allergies and medications related to the injured individual, therefore, the first responder has access to information that is helpful while bringing the identified person to safety. The gathered statistics obtained over time may provide data relating to the accuracy of the initial assessment of the injury. If a new relationship between the initial injury and a predicated issue is obtained and processed at the central repository, then the machine learning model may acquire more training relating to the new relationship to create better prioritization and better predictions.

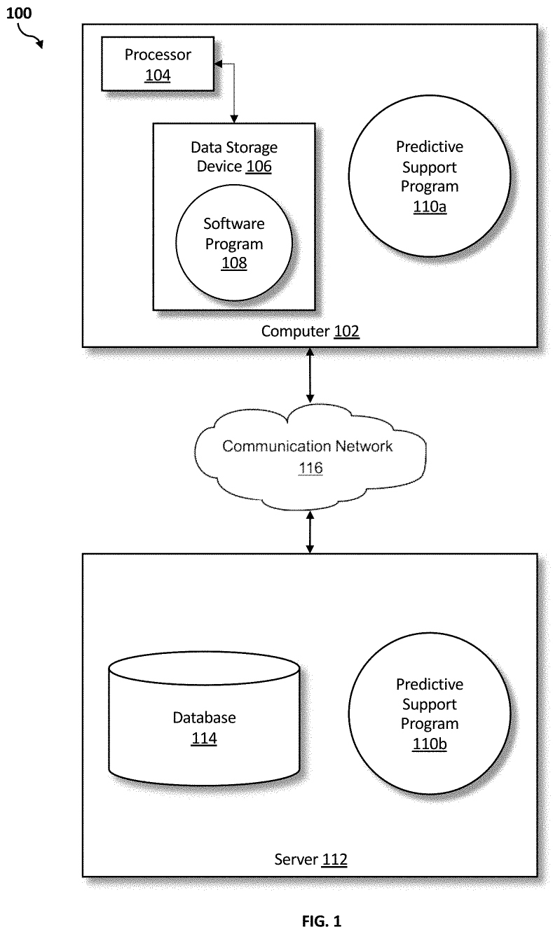

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a predictive support program 110a. The networked computer environment 100 may also include a server 112 that is enabled to run a predictive support program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

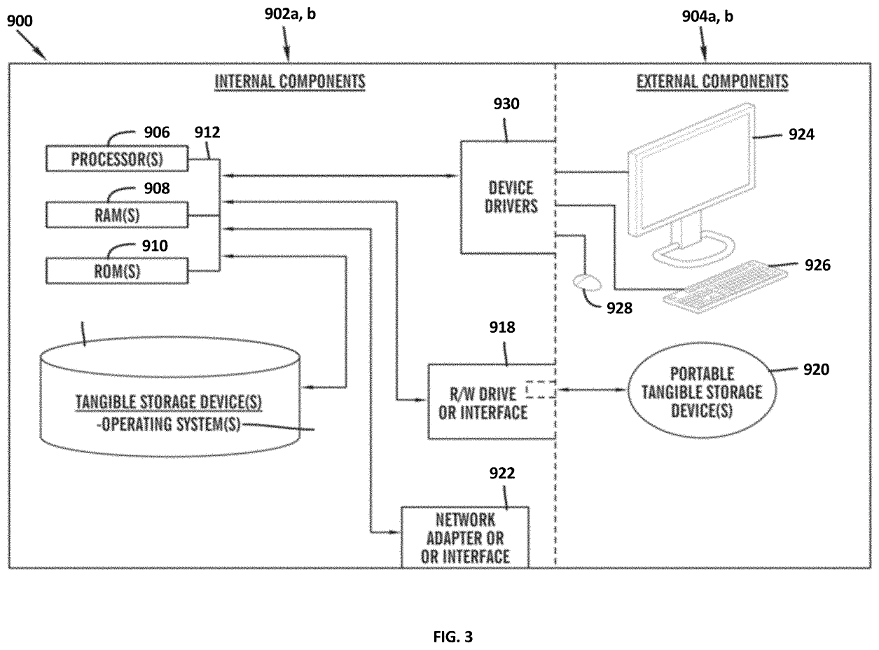

The client computer 102 may communicate with the server computer 112 via the communications network 116. The communications network 116 may include connections, such as wire, wireless communication links, or fiber optic cables. As will be discussed with reference to FIG. 3, server computer 112 may include internal components 902a and external components 904a, respectively, and client computer 102 may include internal components 902b and external components 904b, respectively. Server computer 112 may also operate in a cloud computing service model, such as Software as a Service (SaaS), Analytics as a Service (AaaS), Blockchain as a Service (BaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the predictive support program 110a, 110b may interact with a database 114 that may be embedded in various storage devices, such as, but not limited to a computer/mobile device 102, a networked server 112, or a cloud storage service.

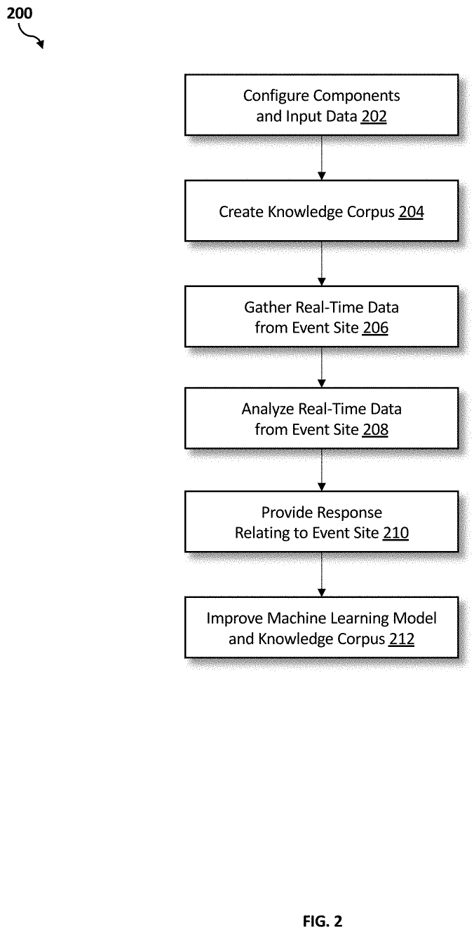

According to the present embodiment, a user using a client computer 102 or a server computer 112 may use the predictive support program 110a, 110b (respectively) to predict the support that may be needed in a location that may not be easily accessible. The predictive support method is explained in more detail below with respect to FIG. 2.

Referring now to FIG. 2, an operational flowchart illustrating the exemplary support prediction process 200 used by the predictive support program 110a, 110b according to at least one embodiment is depicted. The support prediction process 200 may assist various types of personnel in quickly retrieving and recovering data that is helpful during, for example, a crisis situation. The use case example in the present embodiment is based on a natural disaster scenario.

At 202, the components are configured, and the data is imported. Components may include various devices capable of processing, storing and transmitting data to one or more repositories. For example, devices may include wearable devices, computing devices, IoT devices, sensors, smart watches, smart phones, smart tablets, personal computers, augmented reality devices, smart glasses, cameras or microphones. The data that may be stored, transmitted or processed may include audio content, video content, image content, biometric content and textual content. One repository may include a central repository created for a specified domain, for example, a natural disaster knowledge corpus that stores data relating to disaster sites, injuries related to the disaster site and recovery efforts relating to the disaster site.

The components may be configured, for example, using a software application, an IP address, a username and password or an encrypted account. The various devices may be configured and registered to transmit data to and receive data from a central repository or knowledge corpus. The device registration may be created, for example, by a user, an emergency service professional, a medical professional, a SME, a first responder, business personnel, government personnel or administrative personnel. Opt-in and opt-out features are provided and acknowledged in accordance with parameters set during registration. An acknowledgement may be required unless prior consent was given during a pre-registration phase. Alternatively, a user may register a device during, for example, a disaster related event.

At 204, a knowledge corpus is created. The knowledge corpus may include a central repository that collects data relevant to the domain, event, industry or situation. The data collected may include historical data relating to the event or real-time data from an event that is unfolding. The knowledge corpus, for example, relating to a collapsed bridge, may store historical data relating to injuries typical of a similar event, the types of first responders that are typically dispatched, and the types of medical facilities typically needed to treat the injuries. The knowledge corpus may also receive and store transmitted data relating to real-time events. The stored data may be used as training data for the machine learning training of the predictive support program 110a, 110b or for data to use to retrieve a predictive analysis from the predictive support program 110a, 110b. The previously stored data is combined with real-time event data for the current event and over time, the knowledge corpus and the machine learning model builds in accuracy.

At 206, real-time data is gathered from an event site. Real-time data may be collected, for example, at the time a natural disaster is encountered. The collected data may be image data, video data, biometric data, audio data or type-written data. Image data may include pictures taken at the event site, video data may include a video recording or real-time video feed and audio data may include an audio recording or a real-time audio feed. The image, video and audio data may be collected using devices with cameras or microphones. Biometric data may include heartrate data, pupil dilation data, fingerprint data or diabetic data. Biometric data may be collected using devices with, for example, sensors and cameras. Video data may provide information relating to the number of injuries at a disaster site and the types of injures. Audio data may be used to infer the stress levels of the individuals at the disaster site or to be parsed for words and phrases that would infer the severity of injuries encountered. Biometric data may be used to infer the severity of injuries.

At 208, the real-time data is analyzed from the event site. The real-time data is analyzed to provide predictive support relating to an event. For example, if injuries are obtained at a collapsed bridge site, then an analysis of the historical data in the knowledge corpus and machine learning predictive model may provide vital information relating to the disaster site in real-time. Vital information may include data relating to types of injuries, severity of injuries, type of accident, a visual pattern of the injury, previous treatments provided for similar injuries, the response movement pattern of the injured individual and voice textures of the injured individual.

Predictions and real-time data may also be used to prioritize a rescue and treatment process for an injured individual or provide a priority-based list to first responders and emergency personnel relating to treating injured individuals based on severity. Real-time data may be used to map out a disaster site area, to obtain an accurate count of injured individuals without duplicated counting, evaluate the severity of the injuries and to prioritize the evacuation of individuals at the site.

Mapping a disaster site may be provided based on, for example, GPS coordinates, video feeds, audio feeds, social media posts, text messages and phone calls to the police stations. A GPS signal may indicate that a user is within the boundaries related to the disaster site. Images from sensors, cameras or IoT devices may transmit data, such as partitioned data that is viewable from one camera but not others, to a central server and knowledge corpus for processing. The partitioned images may be transmitted from multiple cameras to create a full image of the site. The predictive support program 110a, 110b may mark an area as seen or processed as to avoid overlap or duplicate injury counts. A recount process may occur after a designated amount of time.

At 210, a response relating to the event site is provided. The response may be provided to a person who may be coordinating the activates related to the event site. For example, proper authorities, administrative personnel, first responders, medical personnel, nurses, hospital personnel, fire station personnel, police personnel, users or government authorities. The response may be provided in various formats, such as an alert, a notification, a text-based message, a voice based message or a video based message. The alert may provide information relating to, for example, what to do in the particular disaster scenario to keep safe. A user may be provided messages relating to a particular injury and how to slow down the process of letting the injury worsen. An emergency first responder may be provided with a mapped area of known injured individuals, the names, injuries and medical history for the injured individuals. The hospitals may receive updates relating to how many potential patients to expect within a certain amount of time.

At 214, the machine learning model and the knowledge corpus is improved. The machine learning model may be further trained based on the data obtained during the latest event. For example, results based on the event, the follow-up results, the treatments and the processes used may be fed back into the knowledge corpus for further machine learning and for further model training. The model accuracy may become more accurate with more training and additional follow-up data imported. For example, for a specified time period after the event, follow-up data is imported into the knowledge corpus based on administrative reports, EMS reports, individual reports and medical diagnoses and reports.

It may be appreciated that FIG. 2 provides only an illustration of one embodiment and do not imply any limitations with regard to how different embodiments may be implemented. Many modifications to the depicted embodiment(s) may be made based on design and implementation requirements.

FIG. 3 is a block diagram 900 of internal and external components of computers depicted in FIG. 1 in accordance with an illustrative embodiment of the present invention. It should be appreciated that FIG. 3 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902 a, b and external components 904 a, b illustrated in FIG. 3. Each of the sets of internal components 902 a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the predictive support program 110a in client computer 102, and the predictive support program 110b in network server 112, may be stored on one or more computer-readable tangible storage devices 916 for execution by one or more processors 906 via one or more RAMs 908 (which typically include cache memory). In the embodiment illustrated in FIG. 3, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902 a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the predictive support program 110a, 110b can be stored on one or more of the respective portable computer-readable tangible storage devices 920, read via the respective R/W drive or interface 918 and loaded into the respective hard drive 916.

Each set of internal components 902 a, b may also include network adapters (or switch port cards) or interfaces 922 such as a TCP/IP adapter cards, wireless wi-fi interface cards, or 3G or 4G wireless interface cards or other wired or wireless communication links. The software program 108 and the predictive support program 110a in client computer 102 and the predictive support program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the predictive support program 110a in client computer 102 and the predictive support program 110b in network server computer 112 are loaded into the respective hard drive 916. The network may comprise copper wires, optical fibers, wireless transmission, routers, firewalls, switches, gateway computers and/or edge servers.

Each of the sets of external components 904 a, b can include a computer display monitor 924, a keyboard 926, and a computer mouse 928. External components 904 a, b can also include touch screens, virtual keyboards, touch pads, pointing devices, and other human interface devices. Each of the sets of internal components 902 a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure or on a hybrid cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Analytics as a Service (AaaS): the capability provided to the consumer is to use web-based or cloud-based networks (i.e., infrastructure) to access an analytics platform. Analytics platforms may include access to analytics software resources or may include access to relevant databases, corpora, servers, operating systems or storage. The consumer does not manage or control the underlying web-based or cloud-based infrastructure including databases, corpora, servers, operating systems or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

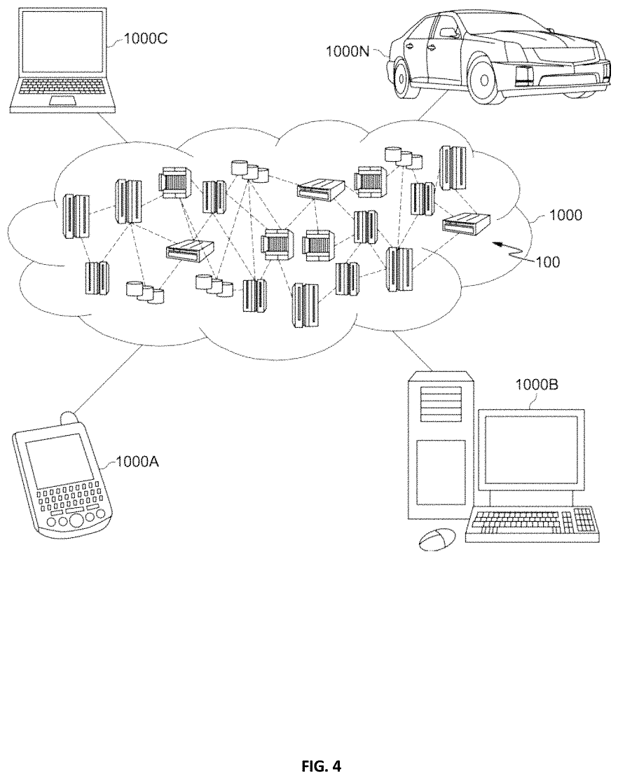

Referring now to FIG. 4, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 4 are intended to be illustrative only and that computing nodes 100 and cloud computing environment 1000 can communicate with any type of computerized device over any type of network and/or network addressable connection (e.g., using a web browser).

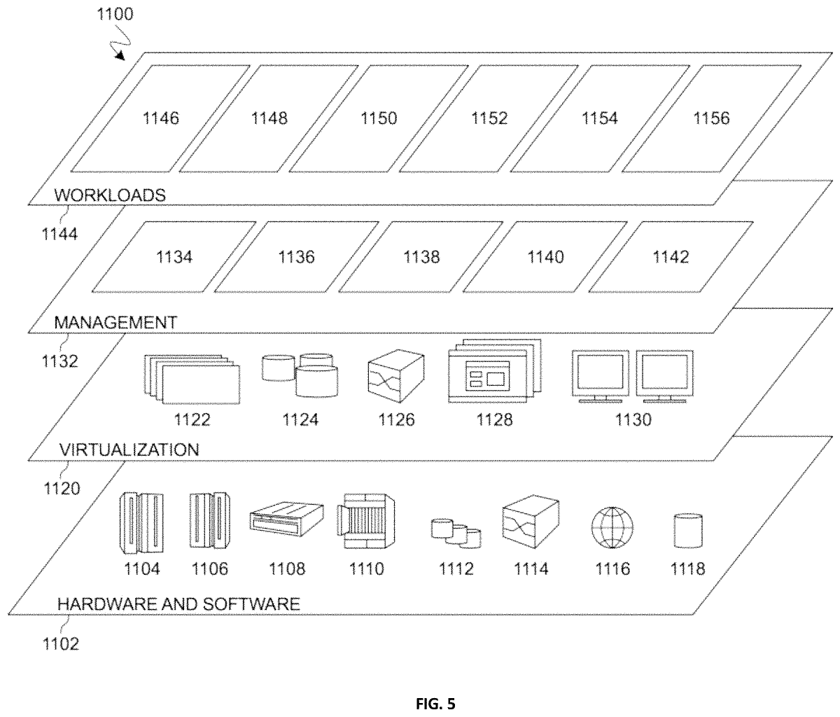

Referring now to FIG. 5, a set of functional abstraction layers 1100 provided by cloud computing environment 1000 is shown. It should be understood in advance that the components, layers, and functions shown in FIG. 5 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and support prediction 1156. A predictive support program 110a, 110b provides a way to assess an event in an area that may be difficult to access.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language, python programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method for predictive support, the method comprising:
creating a knowledge corpus based on historical data;
building a machine learning model using the created knowledge corpus;
gathering real-time data from an event site;
analyzing the gathered real-time data using the built machine learning model;
predicting a response to a plurality of users;
providing the response to the plurality of users; and
training the machine learning model based on the analyzed real-time data.

2. The method of claim 1, wherein the historical data includes domain specific data relating to an event.

3. The method of claim 1, wherein the knowledge corpus includes data gathered from previous similar events, injuries related to similar events and treatments used for previous injuries.

4. The method of claim 1, wherein the real-time data includes image data, video data, biometric data, audio data or type-written data.

5. The method of claim 1, wherein the machine learning model is built based on the historical data.

6. The method of claim 1, wherein the machine learning model is trained further based on the real-time data and ground truth.

7. The method of claim 1, wherein the prediction response includes information related to injuries at the event site and a prioritized list of injured individuals based on a severity level of the injury.

8. A computer system for predictive support, comprising:
one or more processors, one or more computer-readable memories, one or more computer-readable tangible storage media, and program instructions stored on at least one of the one or more computer-readable tangible storage media for execution by at least one of the one or more processors via at least one of the one or more computer-readable memories, wherein the computer system is capable of performing a method comprising:
creating a knowledge corpus based on historical data;
building a machine learning model using the created knowledge corpus;
gathering real-time data from an event site;
analyzing the gathered real-time data using the built machine learning model;
predicting a response to a plurality of users;
providing the response to the plurality of users; and
training the machine learning model based on the analyzed real-time data.

9. The computer system of claim 8, wherein the historical data includes domain specific data relating to an event.

10. The computer system of claim 8, wherein the knowledge corpus includes data gathered from previous similar events, injuries related to similar events and treatments used for previous injuries.

11. The computer system of claim 8, wherein the real-time data includes image data, video data, biometric data, audio data or type-written data.

12. The computer system of claim 8, wherein the machine learning model is built based on the historical data.

13. The computer system of claim 8, wherein the machine learning model is trained further based on the real-time data and ground truth.

14. The computer system of claim 8, wherein the prediction response includes information related to injuries at the event site and a prioritized list of injured individuals based on a severity level of the injury.

15. A computer program product for predictive support, comprising:
one or more computer-readable tangible storage media and program instructions stored on at least one of the one or more computer-readable tangible storage media, the program instructions executable by a processor to cause the processor to perform a method comprising:
creating a knowledge corpus based on historical data;
building a machine learning model using the created knowledge corpus;
gathering real-time data from an event site;
analyzing the gathered real-time data using the built machine learning model;
predicting a response to a plurality of users;
providing the response to the plurality of users; and
training the machine learning model based on the analyzed real-time data.

16. The computer program product of claim 15, wherein the historical data includes domain specific data relating to an event.

17. The computer program product of claim 15, wherein the knowledge corpus includes data gathered from previous similar events, injuries related to similar events and treatments used for previous injuries.

18. The computer program product of claim 15, wherein the real-time data includes image data, video data, biometric data, audio data or type-written data.

19. The computer program product of claim 15, wherein the machine learning model is built based on the historical data.

20. The computer program product of claim 15, wherein the machine learning model is trained further based on the real-time data and ground truth.
